(19) United States
(12) Patent Application Publication
(10) Pub. No.: US 2014/0275873 A1
(43) Pub. Date: Sep. 18, 2014

(54) SENSOR CONNECTOR

(71) Applicant: COVIDIEN LP, Mansfield, MA (US)

(72) Inventors: Timothy W. Fries, Louisville, CO (US); Wanran Ma, Boulder, CO (US)

(73) Assignee: COVIDIEN LP, Mansfield, MA (US)

(21) Appl. No.: 13/803,675

(22) Filed: Mar. 14, 2013

(51) Int. Cl. A61B 5/1455; H05K 1/11

(52) U.S. Cl. CPC A61B 5/14552; H05K 1/11
USPC 600/323; 439/85

(57) ABSTRACT

Embodiments of the present disclosure relate to patient monitoring systems having a connector configured to couple a medical sensor to a monitor. According to certain embodiments, the connector may include a layered printed circuit board having a first surface comprising a plurality of electrical contacts and a second surface having a plurality of electrical contacts. The electrical contacts of the first surface and the electrical contacts of the second surface may be configured to enable the connector to be reversible and to electrically couple the medical sensor to the monitor when the connector is in a first orientation or in a second orientation with respect to the monitor.

### BACKGROUND

The present disclosure relates generally to medical devices and, more particularly, to connectors for coupling a medical sensor to a monitor.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring certain physiological characteristics of a patient. Such devices provide doctors and other healthcare personnel with the information they need to provide the best possible healthcare for their patients. As a result, such monitoring devices have become an indispensable part of modern medicine. For example, photoplethysmography is a common technique for monitoring physiological characteristics of a patient, and one device based upon photoplethysmography techniques is commonly referred to as pulse oximetry. Pulse oximeters may be used to measure and monitor various blood flow characteristics of a patient. A pulse oximeter may be utilized to monitor the blood oxygen saturation of hemoglobin in arterial blood, the volume of individualized blood pulsations supplying the tissue, and/or the rate of blood pulsations corresponding to each heartbeat of a patient. In fact, the “pulse” in pulse oximetry refers to the time-varying amount of arterial blood in the tissue during each cardiac cycle.

A patient in a hospital setting may be monitored by a variety of medical devices, including devices based on pulse oximetry techniques. For example, a patient may be monitored with a pulse oximetry device, which may be appropriate for a wide variety of patients. Depending on the patient's clinical condition, a physician may monitor a patient with a regional saturation sensor placed on the patient's head to determine if the patient is at risk of hypoxia. If a patient is scheduled for surgery, additional or alternative monitoring devices may be applied. For example, one such device may include a sensor for bispectral index (BIS) monitoring to measure the level of consciousness by algorithmic analysis of a patient's electroencephalography (EEG) during general anesthesia.

Various medical devices, such as sensors, are typically coupled to a monitor by a connector. However, each type of medical device and/or each type of monitor typically requires a different type of connector. The many different connectors that are required in the medical setting are inconvenient for the medical practitioner, and the time required to identify and operate the particular connector and/or to learn how to operate the various connectors may result in delays in patient care. Additionally, current connectors must be oriented in a particular way to successfully couple the medical device to the monitor, which is also inconvenient and can lead to delays in patient care.

### BRIEF DESCRIPTION OF THE DRAWINGS

Advantages of the disclosed techniques may become apparent upon reading the following detailed description and upon reference to the drawings in which:

FIG. 1 is a front perspective view of an embodiment of a pulse oximetry monitoring system;

FIG. 2 is a front perspective view of an embodiment of a regional saturation monitoring system;

FIG. 3 is a front view of an embodiment of a bispectral index monitoring system;

FIG. 4 is a top perspective view of an embodiment of a connector and a receptacle for receiving the connector;

FIG. 5 is a side view of the connector and receptacle of FIG. 4 coupled together;

FIG. 6 is a bottom perspective view of an embodiment of a connector and a top perspective view of the receptacle for receiving the connector;

FIG. 7 is a side view of the connector and receptacle of FIG. 6 coupled together;

FIG. 8 is a bottom perspective view of an embodiment of a connector having an integrated circuit chip;

FIG. 9 is a top perspective view of an embodiment of a portion of a connector having a plurality of electrical contacts;

FIG. 10 is a top perspective view of an embodiment of a portion of a connector having a plurality of electrical contacts;

FIG. 11 is a top perspective view of a connector having a housing;

FIG. 12 is a top perspective view of a connector having a latching assembly disposed on top of the connector;

FIG. 13 is a side cross-sectional view of the connector of FIG. 12 coupled to a receptacle;

FIG. 14 is a top perspective view of a connector having a latching assembly disposed on both sides of the connector;

FIG. 15 is a top cross-sectional view of the connector of FIG. 14 coupled to a receptacle; and

FIG. 16 is a side cross-sectional view of a portion of the connector of FIG. 4.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The present disclosure is generally directed to connectors for coupling a medical device to a monitor. The described connectors may be compatible with a variety of medical devices, such as medical sensors, and/or a variety of monitors. For example, the connectors may be utilized to couple pulse oximetry sensors, regional saturation sensors, and/or BIS sensors to various monitors, such as pulse oximeter monitors, regional saturation monitors, BIS monitors, and/or multi-parameter monitors. Additionally, even though the connectors may be used with different medical devices, the described connectors may provide a consistent external appearance and configuration. Further, the connectors described herein may be reversible. That is, the connectors may be configured to electrically couple the medical device to the monitor when the connector is in more than one orientation with respect to the monitor. For example, the connector may be inserted into a corresponding receptacle of the monitor with a top side of the connector facing up or with the top side of the connector facing down.

Connectors in accordance with the present disclosure may provide certain advantages over traditional connectors. For example, connectors that can be used with multiple different types of medical devices may provide cost savings. Additionally, connectors that can connect a medical device to a variety of monitors may provide convenience in the medical setting, as the operator may connect the medical device to a first type of monitor and may conveniently connect the medical device to a second type of monitor should the patient be moved or should a different type of monitor and/or display be desired, for example. Additionally, such connectors may also improve the functionality of patient monitoring systems, as a medical device may be easily coupled to various monitors, which may be configured to process, display, and/or store the data from the sensor in different manners. The uniform external appearance and configuration of the connectors may make the connectors relatively easy to recognize and operate in the fast-paced medical setting. More particularly, such connectors may reduce the amount of time required to couple the medical device to the monitor, as the operator does not have to identify and determine how to operate the particular connector being used. Additionally, medical personnel would not have to learn how to operate or be familiar with numerous different types of connectors. Furthermore, the connector's reversibility, or ability to be inserted into the corresponding receptacle of the monitor in more than one orientation, also may provide convenience and may save time. Such time savings may, in turn, improve patient care.

With the foregoing in mind, FIG. 1 depicts an embodiment of a patient monitoring system 10 that includes a patient monitor 12 that may be coupled to a medical device, such as a sensor 14. In the particular embodiment of FIG. 1, the monitor 12 is a pulse oximetry monitor 12a, and the sensor 14 is a pulse oximetry sensor 14a. The pulse oximetry sensor 14a is connected to the pulse oximetry monitor 12a via a connector 16. The pulse oximetry sensor 14a may include a sensor body 18, which may provide a structural support for the various components (e.g., sensing components) of the pulse oximetry sensor 14a, such as emitters 20 and detectors 22.

The connector 16 may be coupled to the sensor 14 in any suitable manner. For example, the connector 16 may be attached (e.g., permanently affixed) to the sensor 14. In certain embodiments, the connector 16 may be removably (e.g., releasably) coupled to the sensor body 18. Thus, the connector 16 may be a separate component of the monitoring system 10. When the connector 16 is attached or removably coupled to the sensor 14, together the connector 16 and the sensor 14 may form a sensor assembly. Further, as shown, the connector 16 may include a cable 24 that attaches or couples the connector 16 to the sensor 14, providing flexibility between the connector 16 and the sensor body 18, for example. The cable 24 may be of any suitable length to facilitate the coupling of the sensor 14 and the monitor 12 via the connector 16. In certain embodiments as described further below, the connector 16 may not include the cable 24.

Regardless of the manner in which the connector 16 is coupled to the sensor 14, the connector 16 may be configured to fit within a receptacle 26 (e.g., port, aperture, female connector, etc.), which may be disposed in the monitor 12. The receptacle 26 may have a geometry (e.g., a shape, size, configuration) that corresponds to the connector 16 and enables the receptacle 26 to receive at least a portion of the connector 16. In certain embodiments, the monitor 12 may not have the proper receptacle 26 for receiving the connector 16. In such cases, an adapter (e.g., a dongle) may be provided to facilitate coupling of the connector 16 to the monitor 12.

When coupled together, the connector 16 and the receptacle 26 may facilitate the exchange of information between the monitor 12 and the sensor 14. More particularly, sensor 14 may provide electrical signals representative of physiological data to the monitor 12 via connector 16. In some embodiments, the sensor 14 may process the signals and may provide physiological information or parameters to the monitor 12 via the connector 16. Additionally, the monitor 12 may provide instructions and/or operational parameters to the sensor 14 via the connector 16. For example, the monitor 12 may provide settings inputted or selected by the operator using the monitor 12 to the sensor 14 via the connector 16. The monitor 12 may include a monitor display 28 configured to display information regarding the physiological parameters, information about the system, and/or alarm indications, for example. The monitor 12 may also include various input components 30, such as knobs, switches, keys and keypads, buttons, etc., to provide for operation and configuration of the monitor 12 and monitoring system 10. As noted above, the monitor 12 may be configured to receive electrical signals from the sensor 14 via the connector 16, and the monitor 12 may be configured to process the received signals to calculate various physiological parameters, such as oxygen saturation, for example.

The monitor 12 may also be coupled to a multi-parameter monitor 32 via a cable 34 connected to a sensor input port or via a cable 36 connected to a digital communication port. In addition to the monitor 12, or alternatively, the multi-parameter monitor 32 may be configured to calculate physiological parameters and to provide a central display 38 for visualization of information from the monitor 12 and from other medical devices, monitors, and/or or monitoring systems. The multi-parameter monitor 32 may facilitate presentation of patient data, such as pulse oximetry data determined by system 10 and/or physiological parameters determined by other patient monitoring systems (e.g., electrocardiographic (ECG) monitoring system, a respiration monitoring system, a blood pressure monitoring system, etc.). For example, the multi-parameter monitor 32 may display a graph of SpO₂ values, a current pulse rate, a graph of blood pressure readings, an electrocardiograph, and/or other related patient data in a centralized location for quick reference by a medical professional. Although cables 34 and 36 are illustrated, it should be understood that the monitor 12 may be in wireless communication with the multi-parameter monitor 32. Additionally, the multi-parameter monitor 32 may take any suitable form. For example, the multi-parameter monitor 32 may be portable and/or relatively compact. In certain embodiments, the multi-parameter monitor 32 may have all of the functionality of the pulse oximetry monitor 12a, as well as additional functionality of any monitor 12 described herein.

In some embodiments, the multi-parameter monitor 32 may have the receptacle 26 configured to receive the connector 16. In such configurations, the sensor 14 may be directly coupled to the multi-parameter monitor 32 via the connector 16, and the sensor 14 may directly transmit electrical signals to the multi-parameter monitor 32 via the connector 16. In some embodiments, the multi-parameter monitor 32 may include a plurality of receptacles 26 to enable the multi-parameter monitor 32 to be coupled to a plurality of sensors 14 via a plurality of connectors 16 (e.g., a plurality of sensors 14 applied to a patient, a plurality of sensors 14 applied to a variety of patients, and/or a plurality of sensors 14 for the purposes of downloading settings or operational parameters to the different sensors 14, for example, may be connected to the multi-parameter monitor 32 via connectors 16). The connectors 16 and corresponding receptacles 26 may enable various combinations of different types of sensors 14 to be readily and easily coupled the multi-parameter monitor 32 (or other monitor 12) for patient monitoring, without requiring the operator to determine the unique, proper receptacle 26 for each type of connector 16 or for each type of sensor 14, for example.

In certain embodiments, the sensor 14 may not be directly coupled to the monitor 12 during a patient monitoring session, but rather, the sensor 12 may be configured to collect and store data in a memory of the sensor 14 during the patient monitoring session. In some embodiments, the sensor 14 may additionally include a processor configured to process the data, and thus, in certain circumstances, the sensor 14 may calculate and store physiological parameters. In such embodiments, the connector 16 may be used to couple the sensor 14 to the monitor 12 after the patient monitoring session to transfer the stored data or stored calculated parameters from the sensor 14 to the monitor 12. Additionally or alternatively, the connector 16 may be utilized to connect the sensor 14 to the monitor 12 before or after the patient monitoring session to provide or adjust programmed settings, provide instructions or operational parameters, download new software or programs to the sensor 12, or recharge a battery within the sensor 14, for example.

In some embodiments, the sensor 14 may be a wireless sensor 14 that is configured to wirelessly communicate with the monitor 12. Thus, the sensor 14 may wirelessly transmit either raw detector signals or calculated physiological parameter values to the monitor 12 via a wireless module. Additionally, the monitor 12 may use a wireless module to send the sensor 14 instructions and/or operational parameters, such as settings inputted by the operator using the monitor 12. The wireless modules may enable the monitor 12 and the sensor 14 to transmit and/or receive data wirelessly. In such embodiments, the connector 16 may be utilized to connect the wireless sensor 14 to the monitor 12 as a backup method of data transfer. Additionally or alternatively, the connector 16 may be utilized to couple the sensor 14 to the monitor 14 before or after a monitoring session to adjust programmed settings, download new software or programs to the sensor 12, or recharge a battery within the sensor 14, for example. In wireless configurations, the sensor 14 may also include a power source, such as a battery, and appropriate circuitry. Additionally, wireless modules of the sensor 14 and of the monitor 12 may be configured to communicate using the IEEE 802.15.4 standard, and may be, for example, ZigBee, WirelessHART, or MiWi modules. Additionally or alternatively, the wireless module may be configured to communicate using the Bluetooth standard, one or more of the IEEE 802.11 standards, an ultra-wideband (UWB) standard, or a near-field communication (NFC) standard. In such wireless configurations, it may be desirable for the connector 16 to be removably coupleable to the sensor 14, as the wireless sensor 14 is not generally physically connected to the monitor 12 during a patient monitoring session. Further, where the connector 16 is attached to the sensor 14, the connector 16 may have a relatively short cable 24 or no cable 24. Instead, the connector 16 may be attached to the sensor 14 via a flexible connection, may extend directly from the sensor body 18, or may be integrated into the sensor body 18 to facilitate connecting the sensor 14 to the monitor 12 when wired communication is desired.

In embodiments where the sensor 14 is a pulse oximetry sensor 14a, the pulse oximetry sensor 14a may include one or more emitters 20 configured to transmit light. In addition, the pulse oximetry sensor 14a may include one or more detectors 22 to detect light transmitted from the emitters 20 into a patient's tissue after the light has passed through the blood perfused tissue. The detectors 22 may generate a photoelectrical signal correlative to the amount of light detected. The emitter 20 may be a light emitting diode, a superluminescent light emitting diode, a laser diode or a vertical cavity surface emitting laser (VCSEL). Generally, the light passed through the tissue is selected to be of one or more wavelengths that are absorbed by the blood in an amount representative of the amount of the blood constituent present in the blood. The amount of light passed through the tissue varies in accordance with the changing amount of blood constituent and the related light absorption. For example, the light from the emitter 20 may be used to measure blood oxygen saturation, water fractions, hematocrit, or other physiological parameters of the patient. In certain embodiments, the emitter 20 may emit at least two (e.g., red and infrared) wavelengths of light. The red wavelength may be between about 600 nanometers (nm) and about 700 nm, and the IR wavelength may be between about 800 nm and about 1000 nm. However, any appropriate wavelength (e.g., green, yellow, etc.) and/or any number of wavelengths (e.g., three or more) may be used. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure. Additionally, the pulse oximetry sensor 14a may also be configured to monitor various other physiological parameters, such as respiration rate, continuous non-invasive blood pressure (CNIBP), tissue water fraction, hematocrit, and/or water content. The pulse oximetry sensor 14a may include additional functionality, such as temperature or pressure sensing functionality, for example.

As discussed above, the connector 16 of the present disclosure may be configured for use with a variety of medical devices and/or a variety of monitors. For example, as illustrated in FIG. 2, the connector 16 may be utilized to couple a regional saturation sensor 14b to a regional saturation monitor 12b to form a regional saturation monitoring system 40. The regional saturation sensor 14b may have a sensor body 18b that supports various components (e.g., sensing components), such as emitters 20 and detectors 22. For example, the sensor body 18b may support one emitter 20 and two detectors 22 (e.g., a first detector 22a and a second detector 22b). The emitters 20 and detectors 22 may generally have the same light emitting and detecting properties as the emitters 20 and detectors 22 described above with respect to the pulse oximetry sensor 14a. The spacing between the emitter 20 and detectors 22a, 22b enable the collection of oxygen saturation data for the particular region of the body beneath the regional saturation sensor 14b. Although the regional saturation sensor 14b may be configured to be applied to a forehead of the patient to determine the patient's risk of hypoxia, the regional saturation sensor 14b may be configured for placement at any suitable body location.

As shown in FIG. 2, the connector 16 may extend from the sensor body 18b. The regional saturation monitor 12b may include the receptacle 26 to receive at least a portion of the connector 16 to electrically couple the regional saturation sensor 14b to the regional saturation monitor 12b. The regional saturation monitor 12b may be configured to process the signals received from the regional saturation sensor 12b via the connector 16. Additionally, the regional saturation monitor 12b may have a display 28b and inputs 30b. The regional saturation monitor 12b may also be coupled to a multi-parameter monitor 32, as described above with respect to FIG. 1. Additionally, the multi-parameter monitor 32 may have one or more receptacles 26 configured to receive the connector 16, and in such configurations, the regional saturation sensor 14b may be directly coupled to the multi-parameter monitor 32 via the connector 16. In certain embodiments, the multi-parameter monitor 32 may have some or all of the functionality of the regional saturation monitor 12b, as well as additional functionality.

Additionally, as shown in FIG. 3, the connector 16 may be configured to couple a BIS sensor 14c to an EEG monitor 12c (e.g., a BIS monitor) as part of a BIS monitoring system 50. The BIS sensor 14c may include one or more electrodes 52 for collecting an EEG signal, and the BIS monitor 12c may be configured to algorithmically calculate BIS from the EEG signal. As noted above, BIS is a measure of a patient's level of consciousness during general anesthesia. Examples of parameters assessed during the BIS monitoring may include the effects of anesthetics, evaluating asymmetric activity between the left and right hemispheres of the brain in order to detect cerebral ischemia, and detecting burst suppression. Such monitoring may be used to determine if the patient's anesthesia level is appropriate and to maintain a desired anesthesia depth.

As shown, the BIS sensor 14c may be electrically coupled to the BIS monitor 12c via the connector 16. More specifically, the BIS monitor 12c may include the receptacle 26 configured to receive at least a portion of the connector 16. The BIS monitor 14c may include a display 28c and inputs 30c. The display 28c may provide various types of information, such as a patient's BIS value 54, which represents a dimensionless number (e.g., ranging from 0, i.e., silence, to 100, i.e., fully awake and alert) output from a multivariate discriminate analysis that quantifies the overall bispectral properties (e.g., frequency, power, and phase) of the EEG signal. The BIS monitor 12c may also display a signal quality index (SQI) bar graph 56 that indicates the signal quality of the EEG channel source(s), an electromyograph (EMG) bar graph 58 that indicates the power (e.g., in decibels) in the frequency range of 70 to 110 Hz, and a suppression ratio (SR) 60 that represents the percentage of epochs over a given time period (e.g., the past 63 seconds) in which the EEG signal is considered suppressed (i.e., low activity). The BIS monitor 12c may also display the EEG waveform 62 and/or trends 64 over a certain time period (e.g., one hour) for EEG, SR, EMG, SQI, and/or other parameters.

Although not shown in FIG. 3, the BIS monitor 12c may also be coupled to a multi-parameter monitor 32, such as the multi-parameter monitor 28 described above with respect to FIGS. 1 and 2. Additionally, as described above, the multi-parameter monitor 32 may have a receptacle 26 that is configured to receive the connector 16, and in such configurations, the BIS sensor 14c may be directly coupled to the multi-parameter monitor 32 via the connector 16. The multi-parameter monitor 32 may be configured to receive signals receive and/or process signals or parameters received from the BIS sensor 14c. In certain embodiments, the multi-parameter monitor 32 may have some or all of the functionality of the BIS monitor 12c, as well as additional functionality.

FIGS. 1-3 illustrate that the connector 16 may be configured to couple various types of sensors 14 (e.g., pulse oximetry sensors 14a, regional saturation sensors 14b, and/or BIS sensors 12c) to various types of monitors 12 (e.g., pulse oximetry monitors 12a, regional saturation monitors 12b, BIS monitors 12c, and/or multi-parameter monitors 32). However, while examples of some types of sensors 14 and monitors 12, 32 are particularly described above, it should be understood that the connectors 16 may be used to electrically couple a wide variety of sensors 14 to a wide variety of monitors 12, 32.

FIG. 4 is a top perspective view of an embodiment of the connector 16. In the depicted embodiment, the connector 16 includes a printed circuit board (PCB) 70. The PCB 70 may have a first surface 72 (e.g., a top surface) and a second surface 74 (e.g., a bottom surface), and the PCB 70 may extend from a first end 76 to a second end 78 along axis 80. The top surface 72 and the bottom surface 74 may each provide one or more electrical contacts 82 for electrically coupling the sensor 14 to the monitor 12, as described in detail below. When coupled to the sensor 14, the first end 76 may be disposed proximal to the sensor 14, while the second end 78 may be disposed distal from the sensor 14. The cable 24 may extend from the first end 76 of the PCB 70.

As shown in FIG. 4, the connector 16 may have a housing 84 configured to cover (e.g., wrap about, surround, etc.) and/or protect at least part of the PCB 70. More particularly, the housing 84 may cover the first end 76 of the PCB 70, while the second end 78 of the PCB 70 may be exposed (e.g., not covered by the housing 84) to enable the second end 78 to be inserted into the receptacle in the monitor 12 and/or expose the contacts 82. In embodiments including the cable 24, the housing 84 may extend over at least part of the cable 24, thus covering and protecting the connection 86 between the cable 24 and the PCB 70. In certain embodiments, the housing 84 may be overmolded around (e.g., about) at least part of the PCB 70. The housing 84 may be formed from any of a variety of materials, such as elastomers, PVC, silicones, neoprene, isoprene, or any combination thereof. The housing 84 may include a generally soft, rubbery, and/or slightly flexible material, making the connector 16 easy to handle and manipulate. Additionally, the housing 84 may have a surface texture and/or a surface feature (e.g., tab, groove, etc.) to enable the operator to easily and securely grip the connector 16.

As discussed further below, the PCB 70 may have multiple layers (e.g., layers of insulating and conductive materials), each layer extending between the first end 76 and the second end 78 of the PCB 70. The layers may be arranged in an orientation parallel to the top surface 72 and the bottom surface 74. In other words, the layers may be arranged (e.g., stacked) between the top surface 72 and the bottom surface 74. Thus, the PCB 70 may have thickness T that varies based on the number of layers present in the PCB 70. The PCB 70 may include any suitable number of layers. For example, the PCB 70 may include 2, 3, 4, 5, 6, 7, 8, 9, 10, or more layers of insulating and conductive materials, as described in more detail below.

As shown, the PCB 70 may include at least one solder pad 90 (e.g., contacts, conductors, etc.). The solder pads 90 may be disposed in any suitable location, such as proximal to the first end 76 of the PCB 70, and may be covered by the housing 84. Although nine solder pads 90 are illustrated in FIG. 4, the connector 16 may include any suitable number of solder pads 90. For example, 1, 2, 3, 4, 5, 6, 7, 8, 9, 10, 11, 12, 13, 14, 15, or more solder pads 90 may be included in the connector 16. The solder pads 90 are configured to be coupled to the electrical lines (e.g., wires) from the sensor 14. The solder pads 90 are also electrically coupled with a respective contact 82, thus facilitating the transmission of electrical signals from the sensor 14 to the contacts 82. More specifically, wires configured to carry electrical signals from the sensor 14 may be coupled (e.g., soldered) to the solder pads 90. The wires may be routed to the solder pads 90 through the cable 24 of the connector 16. Depending on the type of sensor 14 that is coupled to the connector 16, some or all of the solder pads 90 may be coupled to the wires from the sensor 14. In certain embodiments, another suitable electrical contact may be provided in lieu of solder pads 90, and the wires may be coupled to the contact via any suitable way (e.g., crimping).

The second end 78 of the PCB 70 may be configured to be inserted into the receptacle 26 of the monitor 12 as shown by arrow 92. In other words, the second end 78 of the PCB 70 may form a male connector, and the corresponding receptacle 26 may form a corresponding female connector. As mentioned above, the second end 78 may include electrical contacts 82. Although twelve contacts 82 are illustrated on the top surface 72 of the PCB 70 in FIG. 4, any suitable number of contacts 82 may be provided on the top surface 72 of the connector 16. For example, 1, 2, 3, 4, 5, 6, 7, 8, 9, 10, 11, 13, 14, 15, or more contacts 82 may be provided on the top surface 72 of the connector 16. The number of contacts 82 may be based on the number of electrical signals that are transmitted by the various sensors 14 that are used with the connector 16. Although the number of signals that are transmitted may vary by sensor 14, the connector 16 may provide enough contacts 82 to transmit the signals for the different types of sensors 14. The contacts 82 are configured to align with and engage corresponding receptacle contacts 98 disposed within the receptacle 26. Thus, when the second end 78 of the connector 16 is inserted into the receptacle 26, the contacts 82 are electrically coupled to the receptacle contacts 98, facilitating the exchange of signals and information between the sensor 14 and the monitor 12.

FIG. 5 depicts a side cross-sectional view of the connector 16 of FIG. 4 inserted into the receptacle 26. As shown, second end 78 of the PCB 70 is within the receptacle 26, and the contacts 82 on the top surface 72 of the PCB 70 are electrically coupled to (e.g., in contact with or engaged with) the receptacle contacts 98. The portion of the PCB 70 that is covered by the housing 84 may remain outside of and/or extend from the receptacle 26, enabling the operator to grip and remove the connector 16 from the receptacle 26. The receptacle 26 and/or the PCB 70 may include shielding features, such as one or more metal layers surrounding the receptacle 26 or within the PCB 70.

FIG. 6 is a bottom perspective view of an embodiment of the connector 16. As shown, contacts 82 may be positioned on the bottom surface 74 of the PCB 70. The contacts 82 on the bottom surface 74 may be arranged in the same manner and configuration as the contacts 82 on the top surface 72 of the PCB 70. The contacts 82 on the bottom surface 74 may be electrically-equivalent to the contacts 82 on the top surface 72. In other words, the contacts 82 on the bottom surface 74 may be a mirror-image of the contacts 82 on the top surface 72. Additionally, the contacts 82 on the bottom surface 74 may be configured to align with and engage the receptacle contacts 98 when the connector 16 is inserted into the receptacle 26 with the bottom surface 74 aligned with the receptacle contacts 98, thus facilitating communication between the sensor 14 and the monitor 12.

As described above, and as shown by FIG. 6, the connector 16 is reversible. Thus, the connector 16 may be inserted into the receptacle 26 with the top surface 72 of the PCB 70 aligned with a first side 102 (e.g., top side) of the receptacle 26 as shown in FIG. 4, or the connector 16 may be inserted into the receptacle 26 with the bottom surface 74 of the PCB 70 aligned with the first side 102 of the receptacle 26, as shown by arrow 104 in FIG. 6. Thus, the shape of the PCB 70, the circuitry within the PCB 70, as well as the configuration of the contacts 82 on the top and bottom surfaces 72, 74 of the PCB 70 enable the connector 16 to be inserted into the receptacle 26 in more than one orientation with respect to the receptacle 26 and the monitor 12.

FIG. 7 is a side cross-sectional view of an embodiment of the connector 16 of FIG. 6 inserted into the receptacle 26. The connector 16 is inserted into the receptacle 26 with the bottom surface 74 of the connector 16 aligned with the first side 102 of the receptacle 26. As shown, the second end 78 of the PCB 70 is within the receptacle 26, and the contacts 82 on the bottom surface 74 of the PCB 70 are electrically coupled to (e.g., in contact with or engaged with) the receptacle contacts 98. The portion of the PCB 70 that is covered by the housing 84 may extend from the receptacle 26, enabling the operator to grip and remove the connector 16 from the receptacle 26. As discussed above, the reversibility of the connector 16, as illustrated in FIGS. 4-7, for example, may provide convenience in the medical setting and may save time during patient monitoring sessions, thus improving patient care.

FIG. 8 is a bottom perspective view of an embodiment of the connector 16 coupled to an integrated circuit (IC) chip 110. Although depicted on the bottom surface 74 of the PCB 70, the IC chip 110 may be positioned in any suitable location in or on the connector 16. In the illustrated embodiment, the IC chip 110 is surrounded by the housing 84, which may protect the IC chip 110 from the external environment and/or from operator manipulation. In some embodiments, the IC chip 110 is coupled to one or more solder pads 90, which are in turn electrically coupled to the contacts 82, enabling information from the IC chip 110 to be relayed to the monitor 12 when the connector 16 is inserted into to the receptacle 26. The IC chip 110 may, for example, be soldered directly onto the solder pads 90 or may have one or more extensions to facilitate electrical coupling of the IC chip 110 to one or more of the solder pads 90. As noted above, the solder pads 90 may be electrically coupled with contacts 82, which in turn engage receptacle contacts 98 upon insertion of the connector 16 into the receptacle 26.

The IC chip 110 may serve any of a variety of functions and may be configured to provide various types of information to the monitor 12. For example, the IC chip 110 may enable encryption of sensor data, and may protect against the use of counterfeit sensors 14. More particularly, the IC chip 110 may provide an indication to the monitor 12 that the sensor 14 is compatible and/or that the sensor 14 is genuine. In some embodiments, the IC chip 110 may store information, such as the manufacturer information, that may be communicated to the monitor 12. In some embodiments, the IC chip 110 may additionally or alternatively be configured to store other types of information related to the sensor 14. For example, the IC chip 110 may be a digital memory chip that stores calibration data related to the sensor 14. The IC chip 110 may store information related to the sensor model, type of sensor, the wavelengths of light emitted by the emitters, proper algorithms and coefficients for data processing, and/or instructions or safety messages to be provided to a user via display of the monitor 12, for example. The monitor 12 may also be configured to access and read information stored within the IC chip 110 when the connector 16 is coupled to the receptacle 26. The monitor 12 may display certain types of information on the display or the monitor 12, or the monitor 12 may be configured to provide an alarm if no IC chip 110 is detected within the connector 16, or if the IC chip 110 within the connector 16 cannot be read, for example.

FIG. 9 depicts an alternative configuration of the contacts 82 on the top surface 72 of the PCB 70. As shown, the contacts 82 may be arranged in a single row proximate to the second end 78 of the PCB 70. FIG. 10 depicts yet another alternative configuration of the contacts 82 on the PCB 70. As shown, the contacts 82 may be arranged in three rows proximate to the second end 108 of the PCB 70. Regardless of the configuration of the contacts 82, the configuration of the contacts 82 is the same on the top surface 72 and the bottom surface 74 of the PCB 70 to enable the connector 16 to be reversible. Further, the receptacle 26 of the monitor 12 that receives the second end 78 of the connector 16 will also have a corresponding configuration so that the contacts 82 of the connector 16 will engage the receptacle contacts 98 of the receptacle 26 when the connector 16 and receptacle 26 are mated. Although twelve contacts 82 are provided on the top surface 72 of the PCB 70, any suitable number of contacts 82 may be provided, as noted above. Additionally, although only a few embodiments of the PCB 70 and connector 16 are specifically illustrated, any suitable configuration of the contacts 82 is envisioned. Further, one of skill in the art would recognize that numerous configurations and arrangements of the contacts 82 could be utilized to electrically couple the sensor 12 to the monitor 14, and the illustrated embodiments herein are not intended to be limiting.

In certain embodiments, a tension or force due to frictional contact between the mated portions of the connector 16 and the receptacle 26 may be sufficient to maintain the connector 16 within the receptacle 26 during patient monitoring. Thus, the connector 16 may include a housing 84 having a generally uniform, smooth, and/or featureless surface disposed about the first end 76 of the PCB 70, as shown in FIG. 11. When the connector 16 is inserted into the receptacle 26, only the exposed portion of the PCB 70 is inserted into the receptacle 26, and the housing 84 remains outside of the receptacle 26.

However, in some embodiments, it may be desirable for the connector 16 to have one or more latching assemblies to securely couple the connector 16 to the receptacle 26. For example, such latching assemblies may be desirable as patients are likely to move and change positions, thus pulling on the connector 16 and potentially causing the connector 16 to disengage from the receptacle 26 if sufficient tension or mechanical mating or locking is not provided to hold the connector 16 within the receptacle 26. Any suitable latching assembly may be utilized, such as a mechanical latching assembly.

FIG. 12 depicts a top perspective view of an embodiment of the connector 16 having a latching assembly 120a disposed on a first surface 122 (e.g., top surface) of the connector 16. In certain embodiments, the latching assembly 120a may be part of the housing 84 or may extend from the housing 84. The latching assembly 120a may be the same material, or a different material, as the housing 84. The latching assembly 120a depicted in FIG. 12 may be configured to mechanically secure the connector 16 within the receptacle 26. As shown, the latching assembly 120a includes a protrusion 124 that extends from the top surface 122 of the connector 16. The protrusion 124 may be configured to engage a corresponding feature (e.g., a notch) within the receptacle 26, thus securing the connector 16 within the receptacle 26. The latching assembly 120a may also include a release mechanism 126 configured to release the connector 16 from the receptacle 26, which is described in more detail below. As shown, the release mechanism 126 may have a textured surface to enable the operator to easily grip the release mechanism 126 to release the connector 16 from the receptacle 26.

FIG. 13 depicts a side cross-sectional view of the connector 16 of FIG. 12 secured within the receptacle 26. As shown, the receptacle 26 may include a notch 130 that corresponds with, and is configured to engage, the protrusion 124 of the latching assembly 120a. Thus, when the connector 16 is inserted into the receptacle 26, the protrusion 124 engage the notch 130 to maintain the connector 16 within the receptacle 26, even if tension is applied on the connector 16 by patient movement, for example. As discussed above, the connector 16 may be released from the receptacle 26 by activating (e.g., pressing, engaging, etc.) the release mechanism 126. For example, in the illustrated embodiment, the release mechanism 126 includes a pad, that when depressed (e.g., as indicated by arrow 132), moves (e.g., retracts) the protrusion 124 a sufficient distance to enable the protrusion 124 to clear (e.g., disengage from) the notch 130 so that the connector 16 can be removed from the receptacle 26. Additionally, although shown on the top surface 122 of the connector 16, the latching assembly 120a may alternatively be included on the bottom surface 134 of the connector 16, or two latching assemblies 120a may be provided (e.g., one on the top surface 122 of the connector 16 and one of the bottom surface 134 of the connector 16).

FIG. 14 depicts another embodiment of the latching assembly 120. As shown, the latching assembly 120b may include two arms 140 extending outwardly from the sides of the connector 16 (e.g., one arm 140 may extend from each side of the connector 16). Each arm 140 may have a protrusion 142 that extends outwardly from the arm 140. The protrusions 142 are configured to engage a corresponding feature (e.g., a notch) within the receptacle 26, thus securing the connector 16 within the receptacle 26. Additionally, the arms 140 may extend from or be part of the housing 84 that surrounds at least part of the connector 16. In some embodiments, the connector 16 has a gap 144 between a housing body 146 and each of the arms 140.

FIG. 15 illustrates a top cross-sectional view of the connector 16 of FIG. 14 positioned within the receptacle 26. As shown, the protrusions 142 of the connector 16 may engage with the one or more corresponding notches 152 of the receptacle 26 to retain the connector 16 within the receptacle 26. To release the connector 16 from the receptacle 26, the arms 140 may be motivated (e.g., moved, squeezed, etc.) toward the housing body 144 as indicated by arrows 146. When the arms 140 are moved toward the housing body 144, the protrusions 142 may be moved (e.g., retracted) and may disengage from the notches 152 of the receptacle 26. In an embodiment, the arms 140 are biased outwardly to urge the protrusions 142 into the notches 152. In an embodiment, only one arm 140 is provided on one side of the connector 16. Although FIGS. 12-15 depict various embodiments of latching assemblies 120 having various releasing mechanisms 126, any suitable latching assembly 120 to secure the connector 16 within the receptacle is envisioned. Similarly, any suitable release mechanism 126 for removing the connector 16 from the receptacle 26 may be utilized.

FIG. 16 depicts a side cross-sectional view of a portion of the PCB 70. As discussed above, the PCB 70 may be a multi-layered PCB 70. The contacts 82 may be disposed in a top layer 160 and/or in a bottom layer 162 of the PCB 70. The top layer 160 and the bottom layer 162 may be insulating layers (e.g., may be formed from insulating materials), thus insulating the contacts 82 from one another. The contacts 82 may be flush with the top surface 72 and/or bottom surface 74 of the PCB 70, or the contacts 82 may protrude from the top surface 72 and/or bottom surface 74 of the PCB 70, as shown.

Conductive paths 164 (e.g., traces) may extend between the solder pads 90 and the contacts 82, and the conductive paths 164 may facilitate the transmission of electrical signals to the contacts 82. More particularly, the conductive paths 164 may electrically couple the solder pads 90 to contacts 82 on the top surface 72 and on the bottom surface 74 of the PCB 70 to enable the connector 16 to be reversible. For example, a first solder pad 90 that is electrically coupled to the sensor 14 may be coupled to a first contact 82 on the top surface 72 and a second contact 82 on the bottom surface 74 via conductive paths 164. The conductive paths 164 may be distributed within the layered PCB 70 in any suitable manner, and the conductive paths 164 may be distributed among and between the various layers of the PCB 70 so as to avoid contact between the conductive paths 164. For example, a first conductive path 164a may be disposed along a first intermediate layer 166a and may be coupled to a first contact 82a. A second conductive path 164b may be disposed along a second intermediate layer 166b and may pass through a via 168 to reach the second contact 82b. The via 168 may extend within the second intermediate layer 166b substantially orthogonally with respect to the top surface 72 of the PCB 70. The via 168 may be a passageway (e.g., a channel, a hole) that surrounds the second conductive path 164b, and in some embodiments, the via 168 may have an insulating material that surrounds the second trace 164b. Additionally, a third conductive path 164c may be disposed along a third intermediate layer 166c and may be coupled to a third contact 82c. The third conductive path 166c may pass through a via 168 to reach the contact 82c, as shown. A fourth conductive path 164d may be disposed along a fourth intermediate layer 166d to be coupled to a fourth contact 82d. Although six layers are depicted in FIG. 13, any suitable number of layers may be provided in the PCB 70. For example, in some embodiments, 1, 2, 3, 4, 5, 7, 8, 9, 10, or more layers may be provided.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A connector for coupling a medical sensor to a monitor, the connector comprising:
a layered printed circuit board comprising:
a first surface comprising a plurality of electrical contacts; and
a second surface opposing the first surface and comprising a plurality of electrical contacts, wherein the electrical contacts of the first surface and the electrical contacts of the second surface are configured to enable the connector to electrically couple the medical sensor to the monitor if the connector is inserted into a receptacle of the monitor while in a first orientation and if the connector is inserted into the receptacle of the monitor while in a second orientation.

2. The connector of claim 1, comprising a housing disposed around at least a portion of the connector.

3. The connector of claim 1, comprising a latching assembly configured to releasably secure the connector within the receptacle of the monitor.

4. The connector of claim 4, wherein the latching assembly comprises at least one protrusion extending from a housing disposed around at least a portion of the connector.

5. The connector of claim 1, comprising an integrated circuit chip disposed on the layered printed circuit board.

6. The connector of claim 5, wherein the integrated circuit chip is configured to store data related to the medical sensor.

7. The connector of claim 1, comprising a cable configured to couple the connector to the medical sensor.

8. The connector of claim 7, wherein the cable is configured to be attached to the medical sensor.

9. The connector of claim 1, wherein the medical sensor comprises a pulse oximetry sensor.

10. A system for monitoring a physiological parameter of a patient, the system comprising:
a monitor comprising a receptacle;
a medical sensor; and
a connector configured to couple the medical sensor to the monitor, wherein the connector comprises a plurality of electrical contacts on a first surface and a plurality of electrically-equivalent contacts on a second surface, and the connector is configured to facilitate transmission of electrical signals between the medical sensor and the monitor when the connector is inserted into the receptacle in a first orientation and when the connector is inserted into the receptacle in a second orientation.

11. The system of claim 10, wherein the connector comprises an integrated circuit chip.

12. The system of claim 11, wherein the monitor is configured to read the integrated circuit chip when the connector is positioned in the receptacle.

13. The system of claim 10, wherein the sensor comprises an emitter configured to emit light and a detector configured to detect the light after the light passes through a patient's tissue.

14. The system of claim 13, wherein the monitor comprises a multi-parameter monitor.

15. The system of claim 10, wherein the medical sensor is configured to communicate wirelessly with the monitor.

16. A medical sensor assembly comprising:
a medical sensor comprising:
a sensor body supporting a sensing component configured to sense a physiological parameter of interest when applied to a patient; and
a connector coupled to the sensor body, the connector comprising:
a layered printed circuit board having a top surface comprising a plurality of electrical contacts and a bottom surface comprising a plurality of electrical contacts, wherein the printed circuit board is configured to electrically couple the sensing component to the plurality of contacts on the top surface and to the plurality of contacts on the bottom surface of the printed circuit board.

17. The medical sensor assembly of claim 16, wherein the sensing component comprises an emitter configured to emit light and a detector configured to detect the light after the light passes through a tissue of a patient.

18. The medical sensor assembly of claim 16, wherein the sensing component comprises at least one electrode configured for bispectral index monitoring.

19. The medical sensor assembly of claim 16, wherein the connector comprises a cable configured to couple the connector to the sensor body.

20. The medical sensor assembly of claim 16, wherein the connector is configured to electrically couple the medical sensor to a monitor if the connector is inserted into a receptacle of the monitor in a first orientation or in a second orientation with respect to the receptacle.
